(19) United States
(12) Patent Application Publication
(10) Pub. No.: US 2003/0135265 A1
(43) Pub. Date: Jul. 17, 2003

(54) Prostheses implantable in enteral vessels

(76) Inventor: Jonathan S. Stinson, Plymouth, MN (US)

Correspondence Address: PATENT DEPARTMENT, LARKIN, HOFFMAN, DALY & LINDGREN, LTD., 1500 WELLS FARGO PLAZA, 7900 XERXES AVENUE SOUTH, BLOOMINGTON, MN, 55431, US

(21) Appl. No.: 10/038,640

(22) Filed: Jan. 4, 2002

(51) Int. Cl. A61F 2/06

(52) U.S. Cl. 623/1.16; 623/1.22; 623/1.38

(57) ABSTRACT

A body insertable tubular stent includes discrete tubular segments in an alternating sequence of segments having high axial stiffness and segments having low axial stiffness. The lower axial stiffness segments are intended for placement along more severely curved regions of the body vessel, to provide a greater degree of stent conformity to the vessel. The more axially flexible segments can be provided by winding a metallic or polymeric strand at a higher pitch along such segments, thus to form a higher braid angle in a braided stent. In such cases the more axially flexible segments also exert a higher radial force when the stent is radially compressed. Alternatively, the stent can consist of multiple interbraided strands, with each strand incorporating a higher number of filaments (at least two) along axially stiff segments, and incorporating a lower number of filaments (at least one) along axially flexible segments. In another alternative version, the device is formed of one or more resilient strands, each strand including a biostable filament and a bioabsorbable filament, so that after implantation the radial force and axial stiffness gradually decrease in vivo.

### BACKGROUND OF THE INVENTION

The present invention relates to radially expandable prostheses such as stents and stent-grafts positionable within body lumens, and more particularly to prostheses intended for enteral vessels and other body lumens having natural curvature.

A variety of treatment and diagnostic procedures involve the intraluminal placement and implantation of self-expanding medical prostheses. Such devices are described in U.S. Pat. No. 4,655,771 (Wallsten). The Wallsten devices are tubular structures formed of helically wound and braided strands or thread elements. The strands can be formed of body compatible metals, e.g. stainless steels, cobalt-based alloys or titanium-based alloys. Alternatively the strands can be formed of polymers such as PET and polypropylene. In either event the strands are flexible to permit an elastic radial compression of the prosthesis by its axial elongation.

Typically, a catheter or other suitable delivery device maintains the prosthesis in its radially compressed state when used to intraluminally carry the prosthesis to an intended treatment site for release from the catheter. Upon its release, the prosthesis radially self-expands while its axial length becomes shorter.

The prosthesis is designed to engage surrounding tissue before it expands to its free, unstressed state, thus to provide acute fixation by virtue of an elastic restoring force due to a slight radial compression. The stent or other device may cause a slight radial enlargement of the lumen at the treatment site, yet continue to exert a self-fixating radially outward force, so long as its radius remains smaller than the free-state radius.

For many applications, this self-expanding tendency is considered an advantage as compared to balloon-expandable devices, which typically are made of plastically deformable metals. Further as compared to balloon-expandable devices, a self-expanding device can be deployed without a balloon or other expansion apparatus.

Radially self-expanding prostheses can be provided in configurations other than the interbraided helices discussed in the aforementioned Wallsten patent, e.g. a single coil, or a serpentine configuration of alternating loops designed to allow radial self-expansion without any appreciable axial shortening.

In any event, the radially outward acting restoring force exerted by a given device when compressed to a certain radius less than its free-state radius, depends on the nature of the strands and the device geometry. More particularly, larger-diameter strands, a larger number of strands, and strands formed of a metal or other material having a higher modulus of elasticity, result in a higher level of restoring force. In structures employing helical strands, the restoring force can be increased by increasing the strand crossing angle, i.e. by winding the strands at a lower pitch angle.

Thus, radially self-expanding prostheses can be tailored to facilitate their fixation in a variety of different types and sizes of body lumens. At the same time, practitioners have encountered problems when using these prostheses in body lumens having natural curvature, such as the colon, the duodenum, the iliac and aortic arch, vena caval arch, brachial arch, and fallopian tubes. To illustrate the problem, FIG. 1 schematically shows a curved vessel 1 with an occlusion 2. As seen in FIG. 2, a stent 3 has been deployed within vessel 1 to maintain vessel patency, perhaps after an angioplasty balloon has been used to enlarge the vessel in the area of the occlusion. The radial force exerted by stent 3 is a key factor in maintaining vessel patency and in fixing the stent within the vessel.

FIG. 2 reveals the tendency of stent 3 to straighten the naturally curved vessel, causing a kinking in the vessel (in this case, the colon) near the ends of the stent, as indicated at 4 and 5. The result is an unwanted narrowing of the vessel, and in the case of severe kinking, an obstruction. The source of this problem is the axial stiffness (lack of axial flexibility) of the stent.

The axial stiffness can be reduced by reducing the diameter of the strands making up the stent, using a strand material with a lower modulus of elasticity, or by reducing the number of strands involved. The trouble with these “solutions” is that each reduces the radially outward restoring force exerted by the stent when engaged with surrounding tissue as shown in FIG. 2, with the result that the stent fails to provide the necessary degree of lumen patency and acute fixation.

In connection with prostheses formed of helically wound strands such as stent 3, axial flexibility can be improved by increasing the strand crossing angle of the helices. This approach may appear attractive at first, because the axial stiffness can be reduced without reducing the radially outward restoring force. As noted above, increasing the strand crossing angle increases the radially outward force. The difficulty lies in the fact that as the strand crossing angle increases, so does the extent of stent axial shortening occasioned by a given radial expansion. This increases the need for accurately matching the size of the intended stent with the lumen to be treated, given the increased penalty for excessive radial expansion of the stent once deployed. A related problem is the reduced tolerance for error in axially positioning the stent before its release from the catheter or other deployment device for radial self-expansion.

Therefore, it is an object of the present invention to provide a medical device deployable in a curved body lumen to maintain lumen patency, with axially extending segments of the device individually tailored to support either more gradually curved or more severely curved regions of the lumen.

Another object is to provide a process for fabricating a body insertable tubular structure with discrete axially extending segments in an arrangement of segments with a relatively high axial stiffness alternating with segments with a relatively high axial flexibility.

A further object of the invention is to provide a body insertable prosthesis which, when engaging surrounding tissue after its deployment, provides alternating regions varied selectively as to axial flexibility, radial stiffness, or both.

Yet another object is to provide a process for fabricating a body insertable prosthesis to selectively form discrete segments including at least one segment with relatively high radial force and axial stiffness, and at least one segment with a relatively low radial force and axial stiffness.

### SUMMARY OF THE INVENTION

To achieve these and other objects, there is provided a body insertable prosthesis. The prosthesis is a tubular structure including at least one flexible strand selectively formed to provide a plurality of discrete tubular segments including a first segment, a second segment spaced apart axially from the first segment, and a third segment disposed between the first and second segments. Each of the segments has a nominal diameter when the tubular structure is in a relaxed state, and is radially compressible against an elastic restoring force to a predetermined diameter. The at least one flexible strand further is selectively configured to provide first, second and third axial stiffness levels and radial force levels along the first, second and third segments, respectively, when the segments are radially compressed to the predetermined diameter. The third axial stiffness level is outside of a range of axial stiffness levels bound by the first and second stiffness levels.

In a more basic version of the prosthesis, the first and second segments are at opposite ends, and the third segment provides the intermediate segment of the prosthesis. The intermediate segment can have an axial stiffness level higher than that of the end segments, for example when the treatment site lies along a relatively gradually curved portion of the lumen bound by more severely curved regions. Conversely, when the treatment site lies along a more severely curved lumen region bound by straighter regions, the axial stiffness level of the intermediate segment preferably is less than the axial stiffness level of the end segments.

In another preferred version of the prosthesis, the at least one flexible strand includes a plurality of flexible strands helically wound in opposite directions to form multiple strand crossings defining strand crossing angles. In this embodiment the strand crossing angle of the intermediate segment is outside of a range of strand crossing angles bound by the strand crossing angles of the first and second segments. This embodiment is well suited for applications in which high levels of radial force are required along relatively severely curved sections of the lumen into which the prosthesis is implanted.

If desired, the at least one strand includes a first set of flexible strands spanning substantially the length of the prosthesis, and a second set of flexible strands extending along a first region of the prosthesis to provide a higher axial stiffness and a higher radial force along the first region. A second region of the prosthesis, along which the second set of flexible strands is not provided, has a lower axial stiffness and a lower radial force.

According to another aspect of the present invention, there is provided a body insertable device. The device includes a tubular structure including at least one flexible strand selectively formed to define a plurality of discrete tubular regions of the tubular structure. Along the first region the strand incorporates a first number of filaments. Along the second region, the strand incorporates a second number of filaments less than the first number. As a result, the tubular structure has a first axial stiffness along the first region, higher than a second axial stiffness along the second region.

The tubular structure can be formed by winding one or more flexible strands into the desired tubular shape, wherein each flexible strand is a composite that includes the first number of filaments along its entire length. Then, at least one of the filaments is removed from the tubular structure along the second region, leaving in place the second number of filaments. The filament removal can be accomplished by selective cutting, selective heating to melt or ablate certain filaments at predetermined points, or by selectively dissolving soluble filaments along the second region. When the filaments are to be selectively heated or dissolved, the composite strand includes different types of filaments, with one type that is susceptible to heating or soluable, and another type that is not.

According to another aspect of the present invention, there is provided a prosthesis insertable into body lumens with natural curvature. The prosthesis includes a body insertable tubular wall incorporating an alternating sequence of first and second tubular wall segments including at least three of the wall segments. Each of the wall segments has a nominal diameter when in a relaxed state and is radially compressible against an elastic restoring force to a predetermined diameter. When compressed to the predetermined diameter, the wall segments have respective axial stiffness levels, with each of the first tubular wall segments having a relatively high axial stiffness level and with each of the second tubular wall segments having an axial stiffness level lower than that of the first tubular wall segments. Thus, the second tubular wall segments, as compared to the first tubular wall segments, more readily conform to a curvature of a body lumen in which the tubular wall is deployed.

According to another aspect of the present invention, there is provided a body insertable stent. The stent includes a stent structure formed of at least one flexible composite strand and adapted for deployment at a site within a body lumen to apply a radially outward force against surrounding tissue at the site. The composite strand includes at least one biostable filament and at least one bioabsorbable filament, and is adapted to apply the radially outward force at an initial level upon deployment arising from a combination of the at least one biostable filament and the at least one bioabsorbable filament. The at least one bioabsorbable filament is absorbable in situ, thereby to reduce the radially outward force toward a second level arising from the at least one biostable filament alone.

If desired, the stent structure along its length can include several discrete regions including a first region along which the at least one strand incorporates the at least one biostable filament and the at least one bioabsorbable filament, and a second region along which the at least one strand incorporates only the at least one biostable filament.

The present invention further relates to a process for fabricating a body insertable prosthesis adopted to apply outward radial forces that vary along the prosthesis length, including:

a. providing at least one flexible, biocompatible and thermally formable strand;

b. winding the at least one strand onto a substantially constant diameter cylindrical shaping mandrel, and altering a pitch at least once during winding to form a tubular prosthesis structure having a selected shape in which the at least one strand is wound at a first pitch along a first region of the structure, and wound at a second pitch different from the first pitch along a second region of the structure; and

c. while maintaining the at least one strand in the selected shape, heating the tubular structure to a temperature sufficient to thermally impart the selected shape to the tubular structure.

An alternate process for fabricating a body insertable prosthesis according to the present invention, proceeds with the following steps:

a. winding at least one flexible, biocompatible and thermally formable strand onto a substantially constant diameter shaping mandrel at a substantially uniform pitch, to form a tubular structure;

b. removing the tubular structure from the shaping mandrel, placing the tubular structure onto a heat-set mandrel having a plurality of mandrel segments with different diameters;

c. causing the tubular structure to conform to the heat-set mandrel and thus assume a selected shape in which the tubular structure has a first region with a first diameter, a second region with a second diameter, and an intermediate region between the first and second regions with a third diameter outside of a range of diameters bound by the first and second diameters; and

d. with the tubular structure conforming to the heat-set mandrel, heating the heat-set mandrel sufficiently to thermally impart the selected shape to the tubular structure.

A further process for fabricating a body insertable prosthesis in accordance with the present invention, proceeds as follows:

a. winding at least one flexible, biocompatible and thermally formable strand onto a shaping mandrel having at least a first region with a first diameter and a second region disposed axially of the first region and having a second diameter different from the first diameter, to form a tubular structure having an initial shape;

b. removing the tubular structure from the shaping mandrel, and disposing the tubular structure along and in surrounding relation to a heat-set mandrel;

c. causing the tubular structure to substantially conform to the heat-set mandrel, thereby to assume a selected shape; and

d. with the tubular structure in the selected shape, heating the tubular structure to a temperature sufficient to thermally impart the selected shape to the tubular structure and thereby form in the tubular structure first and second segments corresponding respectively to portions of the tubular structure previously disposed along the first and second regions of the shaping mandrel, the first and second segments being adapted to exert respective first and second different levels of radially outward force when the tubular structure is radially compressed to a predetermined diameter.

The heat-set mandrel can have a uniform diameter, or alternatively can include discrete sections with different diameters.

Further according to the present invention, there is provided a process for fabricating a body insertable prosthesis with segments that differ in axial stiffness and radial force. The process includes:

a. providing a flexible strand that is a composite of at least two body compatible filaments;

b. selectively winding the strand to form a tubular structure with a selected shape; and

c. selectively removing at least one of the filaments from the at least one strand along a predetermined axially extended region of the tubular structure, whereby the tubular structure along the predetermined region has a reduced axial stiffness level and a reduced radial force level as compared to a remaining region of the tubular structure.

In accordance with the present invention, a medical device deployable in a curved body lumen to maintain lumen patency includes axially extending segments individually tailored to support either more gradually curved or more severely curved regions of the lumen. Segments with higher axial stiffness can be provided along relatively straight regions of the lumen, with segments having relatively high axial flexibility provided along the more severely curved regions. The devices can incorporate segments with different pitch angles or braid angles, whereby segments with higher axial flexibility also exert higher levels of radial force. The devices can be formed of strands that incorporate different numbers of filaments along different segments, such that the more axially flexible segments exert lower levels of radial force. Strands incorporating several filaments also can incorporate at least one bioabsorbable filament, providing levels of radial force and axial stiffness that decrease in situ.

### IN THE DRAWINGS

For a further understanding of the above and other features and advantages, reference is made to the following detailed description and to the drawings, in which:

FIG. 1 is a schematic view of a curved body vessel with an occlusion;

FIG. 2 is a schematic view of a conventional stent implanted in the vessel;

FIG. 3 is a schematic view of a stent, fabricated according to the present invention, implanted in a curved body vessel, a portion of which is cut away to reveal the stent;

FIG. 4 is an elevational view of another stent constructed in accordance with the present invention;

FIG. 5 is a side elevation of an alternative embodiment stent;

FIG. 6 illustrates the stent of FIG. 5 implanted in a body vessel;

FIG. 7 is a side elevation of a further alternative embodiment stent;

FIG. 8 is an enlarged partial view of the stent in FIG. 7, showing a single strand of the stent;

FIG. 9 is a side elevation of a further alternative embodiment stent;

FIG. 10 is a side elevation of the stent shown in FIG. 9, after a bioabsorbable constituent of the stent has been absorbed in situ;

FIG. 11 is a side elevation of an alternative embodiment stent composed of a single strand;

FIGS. 12 and 13 illustrate stages in a process for fabricating a stent similar to that shown in FIG. 4;

FIGS. 14 and 15 illustrate stages of a process for fabricating a stent similar to that shown in FIG. 5;

FIG. 16 illustrates a braiding stage of a process for fabricating a stent similar to that shown in FIG. 5;

FIGS. 17 and 18 illustrate stages in a process for fabricating a stent similar to that shown in FIG. 7; and

FIG. 19 illustrates a stage of a process for fabricating a stent similar to that shown in FIG. 7.

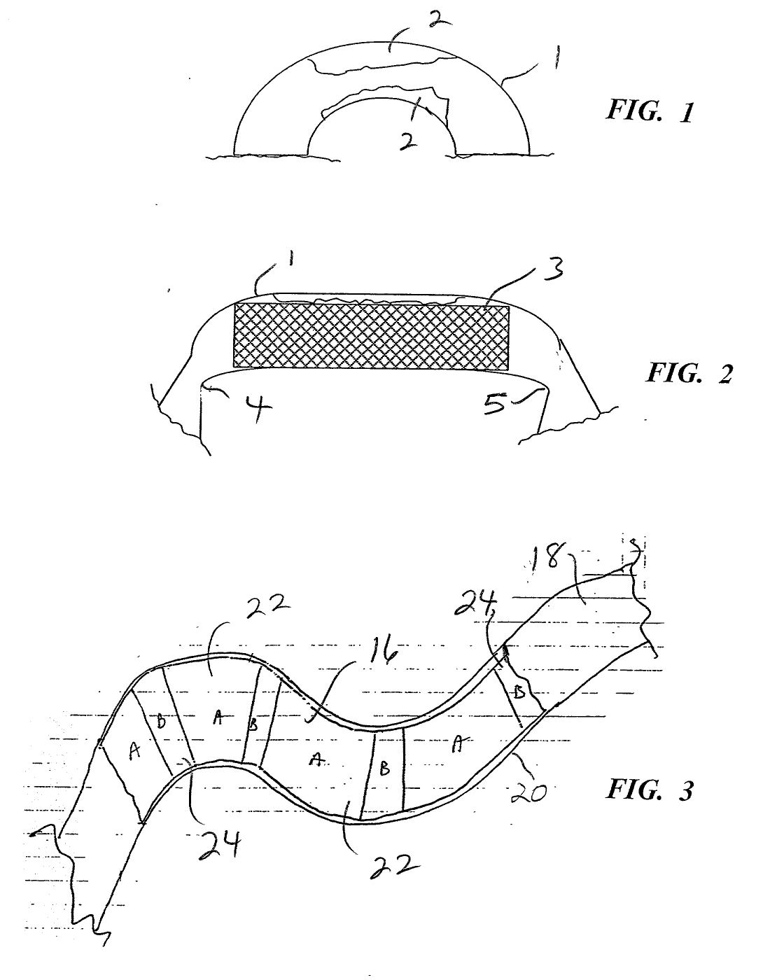

### DETAILED DESCRIPTION OF THE PREFERRED EMBODIMENTS

Turning now to the drawings, there is shown in FIG. 3 a body insertable stent 16, implanted within a body vessel having natural curvature, in particular the colon 18. The colon has a tissue wall 20 which surrounds the stent over its entire length, although part of the tissue wall is cut away in the figure to reveal the stent.

Stent 16 is a radially self-expanding stent, formed of helically wound, interbraided flexible strands. Consequently the stent is deformable elastically to a reduced-radius, extended-length configuration to facilitate its intraluminal delivery to the treatment site in the colon. Once at the treatment site, the stent is released from a catheter or other delivery device (not shown) and radially self-expands into an engagement with tissue wall 20. When engaged with the wall, stent 16 is maintained at a predetermined diameter less than a diameter of the stent when in a free state, i.e. when subjected to no external stress. Accordingly the stent as shown in FIG. 3 retains an internal elastic restoring force, and exerts this force radially outwardly against tissue wall 20. This force acts against obstructive tissue to maintain a patency of the lumen within the colon. The force also tends to embed the stent strands into the vessel wall, and thus prevent stent migration. Meanwhile, tissue wall 20 exerts a counteracting radially inward force to contain the stent.

Stent 16 is composed of a plurality of discrete segments, including two different types of segments 22 and 24 respectively labeled “A” and “B” in the figure. The stent segments have different properties when stent 16 is maintained at the predetermined diameter as shown. In particular, stent segments 22, as compared to stent segments 24, have a higher axial or longitudinal stiffness. Segments 24 have higher axial flexibility, and accordingly more readily conform to the curvature of colon 18. Stent segments 22 mitigate the axial foreshortening of the stent, as the stent radially self-expands after its release from a deployment device.

As seen in FIG. 3, segments 22 and 24 are arranged in an alternating sequence. Stent segments 22 are disposed along the straighter, more gradually curved regions of colon 18, while stent segments 24 are positioned along the more severely curved sections of the vessel. Accordingly, segments 22 and segments 24 cooperate to provide the stent fixation force. Segments 24 avoid the kinking problem that arises when a stent with excessive axial stiffness tends to straighten a naturally curved region of the colon or another vessel. Segments 22 serve to dilute the overall stent foreshortening that would complicate positioning of the stent across a lesion during deployment.

As shown in FIG. 3, segments can vary as to their lengths. In some applications, segments 22 are longer than segments 24 as shown, while in other applications segments 24 are longer. Although only two types of segments are shown, it is contemplated within the present invention to provide segments exhibiting three or more different levels of radial force or axial stiffness.

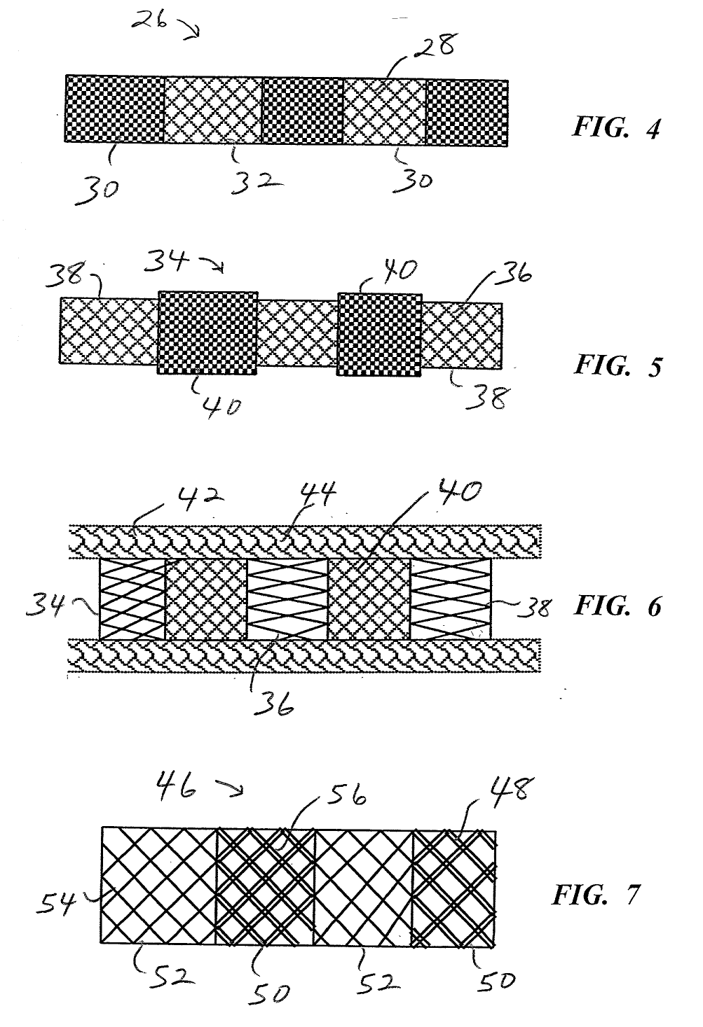

FIG. 4 illustrates an alternative embodiment stent 26 formed by helically winding and interbraiding multiple elastic biocompatible strands 28. The strands as flexible, preferably formed of a cobalt based alloy sold under the brand name Elgiloy. Suitable alternative metals include stainless “spring” steel and titanium/nickel alloys. Certain polymers as also suitable, including PET, polypropylene, PEEK, HDPE, polysulfone, acetyl, PTFE, FEP, and polyurethane.

Stent 26 consists of an alternating sequence of segments 30 in which the strands form a crossing angle of 150 degrees, and segments 32 in which the strands define a crossing angle of 130 degrees. The strand crossing angle or braid angle is conveniently thought of as an angle bisected by a plane incorporating a longitudinal central axis of the stent, i.e. a horizontal axis as viewed in FIG. 4. The pitch angle is the angle at which the strands are wound with respect to a plane normal to the axis. Thus, the pitch angles of segments 30 and 32 are 15 degrees and 25 degrees, respectively. In FIG. 4a, “p” indicates the pitch angle and “α” indicates the strand crossing angle.

The higher strand crossing angle in segments 30 results in a higher level of radially outward force exerted by these segments, as compared to segments 32, when the stent is maintained at the predetermined diameter. Segments 30 also have a higher axial flexibility. Conversely, segments 32 undergo less axial shortening when stent 26, upon its release from a deployment device, radially self-expands. Thus, in stent 26 the end segments and central segment provide the greater stent fixation force, yet more readily conform to any curvature of the vessel in which stent 26 is implanted. This configuration is particularly useful in cases where occlusions are present in more severely curved regions of the vessel.

FIG. 5 shows an alternative embodiment stent 34 which, like stent 26, is formed of helically wound and interbraided flexible and biocompatible strands 36. The stent provides an alternating sequence of stent segments 38 with a strand crossing angle of 130 degrees, and stent segments 40 with a strand crossing angle of 150 degrees. In addition, segments 40 are formed to have larger diameters than segments 38 when stent 34 is in the free state.

In FIG. 6, stent 34 is shown after its deployment within a vessel 42 defined by a tissue wall 44. Although vessel 42 actually is curved, it is shown in straight lines in FIG. 6 to draw attention to the manner in which stent 34 is radially compressed and thereby maintained at a predetermined diameter. In particular, segments 40, while larger than segments 38 in the free state, are radially compressed to the predetermined diameter along with segments 38. Accordingly, as compared to segments 30 of stent 26 and assuming other structural parameters are equal, segments 40 exert a higher level of radially outward force to fix the stent and enlarge an obstructed region of the vessel. As before, segments 40 of stent 34 have higher axial flexibility, and thus more readily conform to vessel curvature.

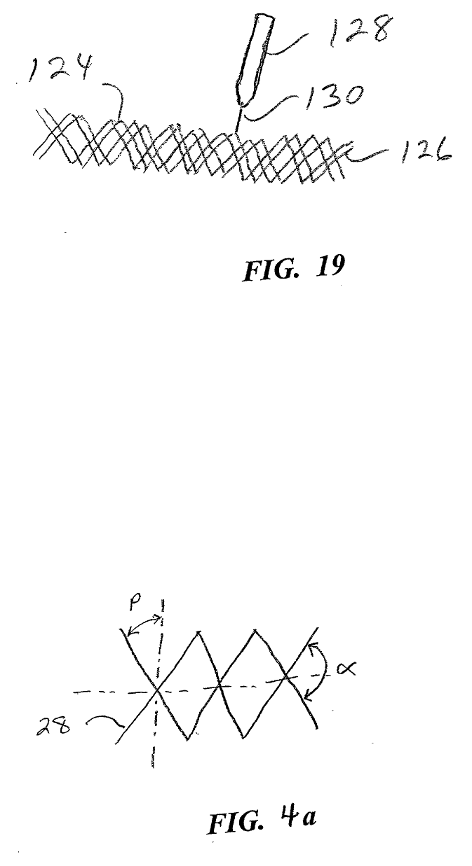

FIG. 7 illustrates another alternative embodiment stent 46 formed of flexible, helically wound and interbraided strands 48. The strands are wound to provide a strand crossing angle that remains substantially constant over the entire stent length, so that alternating stent segments 50 and 52 have the same strand crossing angle.

Segments 50 and 52 are distinguished from one another, based on the makeup of the strands. In particular, along segments 50, each of the strands incorporates two filaments, indicated at 54 and 56. Along segments 52, however, each strand incorporates only filament 54. With filaments 54 and 56 contributing to both the radial force and axial stiffness along segments 50, these segments exert higher levels of radial force, and have higher levels of axial stiffness, as compared to segments 52.

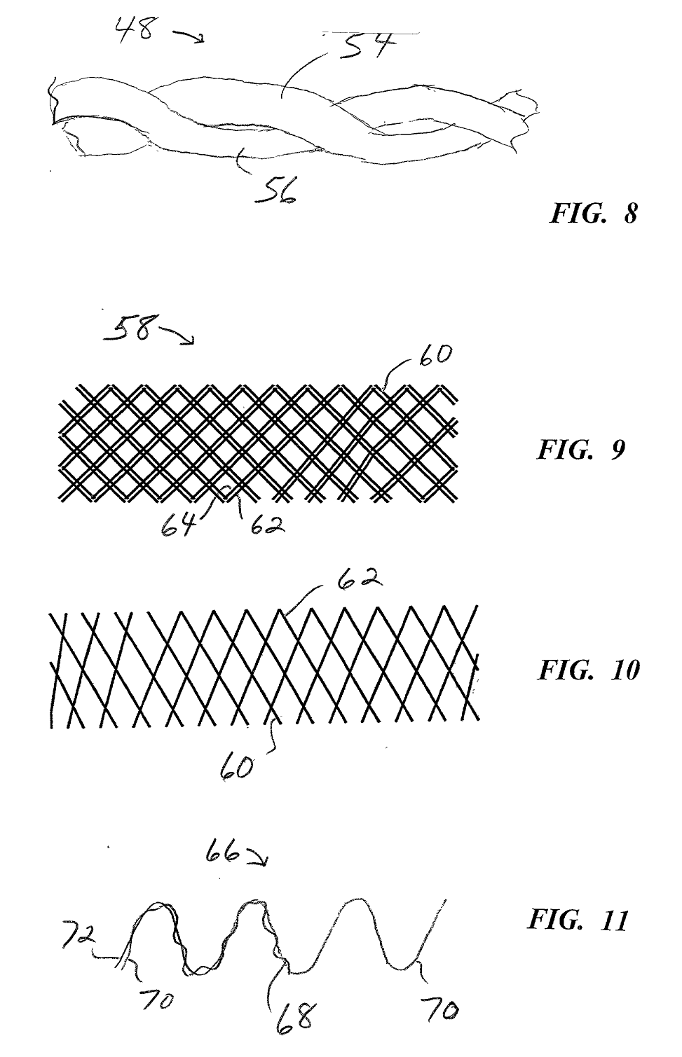

Along segments 50, filaments 54 and 56 may simply be provided side-by-side, and braided in a one pair over-one pair under pattern. In more preferred versions, filaments 54 and 56 are at least slightly twisted into the form of a rope or cable as seen in FIG. 8. Filaments 54 and 56 can have the same diameter as shown. However, to increase the contrast between the radial force and axial stiffness exhibited by segments 50 as compared to segments 52, filament 54 can have a larger diameter then filament 56. To reduce the contrast, filament 56 can be provided with the larger diameter. Similar results may achieved by providing more than two filaments in each strand. For example, if the strand along segments 50 is composed of three filaments, the strand along segments 52 can consist of either one or two of the filaments, for a greater or lesser contrast between alternating segments.

A wide latitude of control over stent characteristics is afforded by selecting filament materials. In the version of stent 46 shown in FIGS. 7 and 8, both filaments are wires, formed for example of the previously mentioned Elgiloy alloy or stainless spring steel. In another version of this stent, both filaments are formed of a polymer such as PET.

Yet another alternative is to employ filaments formed of a bioabsorbable material from which flexible filaments can be formed. Suitable materials include poly (alpha-hydroxy acid) such as polylactide [poly-L-lactide (PLLA), poly-D-lactide (PDLA)], polyglycolide (PGA), polydioxanone, polycaprolactone, polygluconate, polylactic acid-polyetholene oxide copolymers, poly (hydroxybutyrate), polyanhydride, polyphosphoester, poly(amino acid), and related copolymers. These materials have characteristic degradation rates in vivo. PGA is bioabsorbed relatively quickly, for example in a matter of weeks to months. By contrast, PLA is bioabsorbed over a period of months to years.

In any event, the strands preferably incorporate both bioabsorbable and biostable filaments. Once a stent employing these filaments is implanted, the bioabsorbable filaments begin to degrade, whereby the radial force of the stent and the axial stiffness of the stent gradually decrease in situ, eventually to a point where the stent radial force and axial stiffness, along all segments, is equivalent to the radial force and axial stiffness of a stent incorporating only the biostable filaments. In one specific example of this version of stent 46, filament 54 is formed of PET (Dacron), while filament 56 is formed of one of the bioabsorbable materials mentioned above.

In further versions of stent 46, filament 54 is a wire formed of a metal such as the Elgiloy alloy, and filament 56 is formed of a polymer such as PET.

In general, using different materials to form filaments 54 and 56 enables fabrication of stent 46 using processes other than those available when all filaments are the same kind. In all of the prostheses, strands are formed first by winding all of the intended filaments into strands, after which the strands are interbraided to form an intermediate tubular structure in which each strand is uniform over its entire length. Then, segments 52 are formed by selectively removing portions of filament 56 (or filaments 56 in the case of multiple-filament strands) along a region of the stent corresponding to segments 52. When filaments 54 and 56 are the same material, this selective removal is accomplished by cutting each filament 56 at selected points along the stent length to separate the portions of each filament 56 intended for removal. The cutting and removal is labor-intensive and costly.

When filament 54 is metal and filament 56 is polymeric, the polymeric filaments are susceptible to heat treatments or chemical treatments that have negligible impact on the metallic filaments. Accordingly, the polymeric filaments can be removed by laser ablation or other exposure to heat, or dissolved, as explained in greater detail below.

FIG. 9 shows a further alternative embodiment stent 58 formed by helically winding and interbraiding multiple flexible biocompatible strands 60. Each of the strands is composed of two filaments: a filament 62 formed of a biostable polymer such as PET; and a filament 64 formed of a bioabsorbable polymer. Stent 58 is particularly useful in circumstances where the physician desires to implant a stent that initially exerts a high radial force and has a high axial stiffness, but also expects that after a predetermined time, e.g. about a month, the stricture will be remodeled and the initial radial force and axial stiffness levels will no longer be necessary. Once stent 58 is implanted, its radial force and axial stiffness diminish gradually in vivo, due to the degradation of the bioabsorbable polymer. Eventually, substantially all of bioabsorbable filaments 64 are absorbed, leaving a stent 58 composed of strands 60 consisting essentially of filaments 62, as seen in FIG. 10. Another important advantage is that the pitch or strand crossing angles can be kept low, reducing the amount of axial contraction as the stent radially expands during deployment from the catheter.

FIG. 11 illustrates a further alternative embodiment stent 66, formed of a single, helically wound strand 68. Along a first segment of the stent, the strand incorporates filaments 70 and 72, preferably twisted in the form of a cable or rope as illustrated in FIG. 8. Along a second segment of the stent, strand 68 incorporates only filament 70. Filament 70 can be formed of metal, or a biostable polymer. Filament 72 can be formed of a metal, a biostable polymer, or a bioabsorbable polymer. Strand 66 can incorporate multiple filaments 70 and multiple filaments 72, if desired.

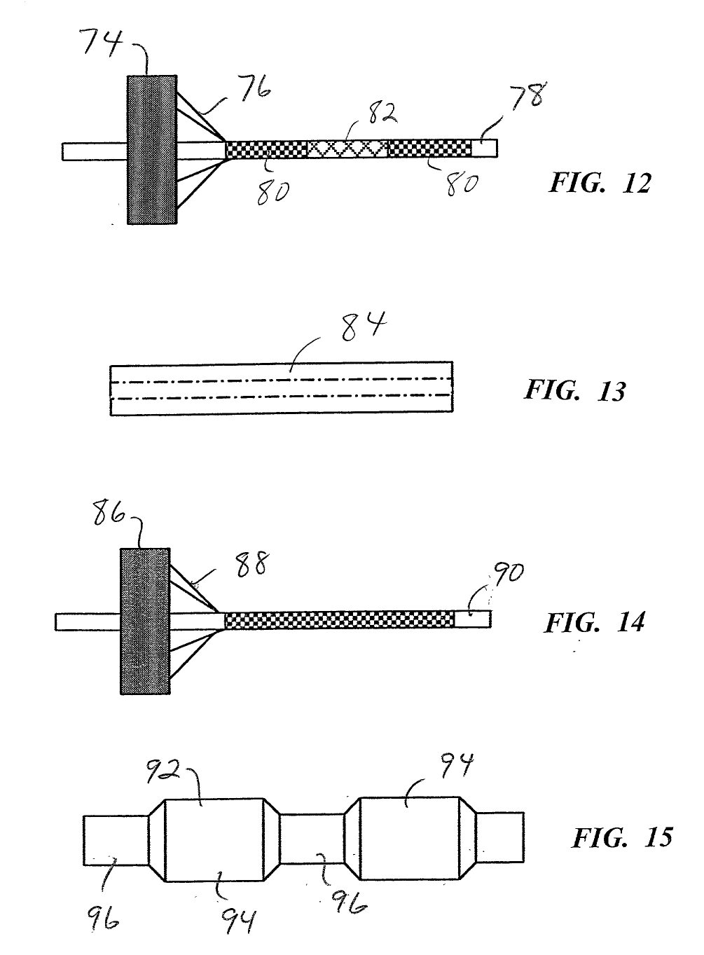

A variety of processes can be employed for fabricating stents with axial stiffness levels and radial stiffness levels that vary selectively along the stent length. FIG. 12 illustrates a braiding apparatus 74 used to simultaneously wind and interbraid a plurality of strands 76 onto a shaping mandrel 78. While just a few strands are illustrated, an exemplary process can involve 36 strands, i.e. 36 separate bobbins (not shown) from which the strands are played out simultaneously.

During winding, the helical pitch of the strands is changed periodically to provide an intermediate tubular structure with segments such as shown at 80 in which the strand crossing angle is 150 degrees, alternating with segments 82 having a strand crossing angle of 130 degrees.

The tubular structure is removed from shaping mandrel 78, and placed over a tubular heat-set mandrel (FIG. 13), which has a constant diameter, preferably the same as the diameter of the shaping mandrel. Mandrel 84 is heated sufficiently to raise the temperature of the tubular structure at least to a heat-set temperature to thermally impart the desired shape, resulting in a device like stent 26 (FIG. 4). Table 1 lists structural characteristics for a stent of this type, in which 36 Elgiloy strands, each having a diameter of 0.17 mm, are wound on a 22 mm diameter shaping mandrel and thermally formed on a 22 mm diameter heat-set mandrel. The pressure and stiffness levels in Table 1 characterize the stent when radially compressed to a diameter of 22 mm.

TABLE 1

| Strand Crossing | | Longitudinal |
|---|---|---|
| Angle, Degrees | Radial Pressure, Pa | Stiffness, N/m |
| 50 | 14 | 34.1 |
| 70 | 60 | 30.1 |
| 100 | 314 | 22.6 |
| 125 | 886 | 16.4 |
| 130 | 1,280 | 14.3 |
| 135 | 1,388 | 13.4 |
| 140 | 2,138 | 10.5 |
| 150 | 2,992 | 8.6 |
| 155 | 3,198 | 7.5 |

table 1 illustrates the general point that stent segments wound at higher strand crossing angles have higher axial flexibility (i.e. lower longitudinal stiffness) and exert higher levels of radially outward force when radially compressed. With particular attention to stent 26, stent segments 30 wound at 150 degrees, as compared to stent segments 32 wound at 130 degrees, exert more than twice the radially outward force (2,992 Pa compared to 1,280 Pa) and have slightly over half the longitudinal stiffness. A tolerance of 5 degrees in the strand crossing angle results in considerable ranges of levels encompassing the nominal radial pressure in particular, but also the nominal longitudinal stiffness.

The present invention encompasses alternative processes. FIGS. 14 and 15 relate to a process for fabricating stent segments with different strand crossing angles in which the control parameters of the braiding equipment remain constant. As a result, the initial braided structure has a constant crossing angle over its complete length. More particularly, FIG. 14 shows a braiding apparatus 86 used to simultaneously wind and interbraid a plurality of strands 88 onto a constant-diameter shaping mandrel 90. The resulting intermediate tubular structure has a constant strand crossing angle.

The tubular structure is removed from the shaping mandrel and placed onto a heat-set mandrel that does not have a constant diameter, such as a heat-set mandrel 92 shown in FIG. 15, with larger-diameter sections 94 alternating with smaller-diameter sections 96. With the tubular structure surrounding mandrel 92, its opposite ends are pulled away from one another to radially contract the structure and draw it into a closer engagement with mandrel 92. Then, clamps (not shown) are closed against the tubular structure, causing it to more closely conform to the contour of the mandrel.

In one specific example, shaping mandrel 90 has a diameter of 23 millimeters, sections 94 of the heat-set mandrel have a diameter of 23 millimeters, and sections 96 of the heat-set mandrel have a diameter of 22 millimeters. The strand crossing angle of the tubular structure is 150 degrees. When drawn around and clamped against the heat-set mandrel, portions of the tubular structure along mandrel sections 94 assume substantially the same diameter at which the structure was shaped. As a result, the strand crossing angle remains at about 150 degrees. Along smaller mandrel sections 96, the tubular structure is contracted to a smaller diameter of 22 millimeters, with a result that the strand crossing angel is reduced to about 130 degrees.

With the tubular structure conforming to the heat-set mandrel, heat from the mandrel raises the structure temperature at least to a heat-set temperature, whereby the mandrel shape is thermally imparted to the structure. The result is a stent similar to stent 34 (FIG. 5), with three smaller-diameter stent segments 38 with a strand crossing angle of 130 degrees, and two larger-diameter stent segments 40 with a strand crossing angle of 150 degrees.

Table 2 lists various structural parameters for a stent formed of 36 Elgiloy alloy strands having a diameter of 0.17 millimeters, for a variety of different mandrel diameters and strand crossing angles. Table 2 illustrates the impact upon crossing angle when the structure is drawn to a reduced diameter, and also indicates resulting pressure and stiffness levels.

TABLE 2

| Shaping | Initial | | Post Heat-Set | | |
|---|---|---|---|---|---|
| Mandrel | Crossing | Heat-Set | Crossing | | Longitudinal |
| Diameter, | Angle, | Mandrel | Angle, | Radial | Stiffness, |
| mm | Degrees | Diameter, mm | Degrees | Pressure, Pa | N/m |
| 24 | 130 | 22 | 112 | 561 | 19.3 |
| 24 | 130 | 24 | 130 | 1,239 | 16.1 |
| 25 | 130 | 22 | 106 | 421 | 21 |
| 25 | 130 | 25 | 130 | 1,154 | 17 |
| 24 | 140 | 22 | 119 | 778 | 17.4 |
| 24 | 140 | 24 | 140 | 1,741 | 12.9 |
| 22 | 130 | 22 | 130 | 1,280 | 14.3 |
| 22 | 130 | 21 | 116 | 494 | 17 |
| 23 | 150 | 22 | 135 | 1,595 | 12.8 |
| 23 | 150 | 23 | 150 | 2,679 | 9.2 |

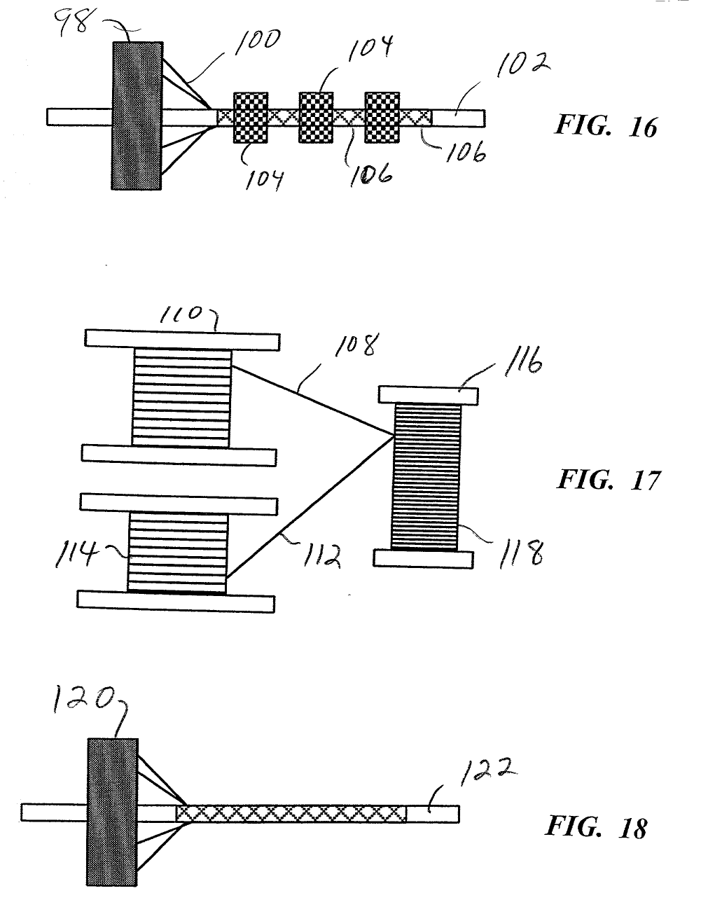

FIG. 16 illustrates an alternative embodiment of the preceding process, in which a braiding apparatus 98 is used to wind and interbraid a plurality of strands 100 onto a shaping mandrel 102. Mandrel 102 has a plurality of larger-diameter sections 104 in an alternating sequence with smaller-diameter sections 106. The resulting intermediate tubular structure includes alternating large-diameter segments and smaller-diameter segments. Strands 100 are wound to provide a strand crossing angle that is constant over the length of the intermediate tubular structure. Alternatively, the strands can be wound to provide higher strand crossing angles along the larger-diameter segments, if desired.

According to one version of this process, the intermediate tubular structure is removed from shaping mandrel 102 and disposed about a heat-set mandrel similar to mandrel 92 shown in FIG. 15, with alternating larger-diameter and smaller-diameter sections corresponding to the same sections of the shaping mandrel. Sufficient heat is applied to thermally impart the desired shape to the tubular structure, resulting in a stent similar to stent 34 (FIG. 5).

According to another version of this process, strands 100 again are wound about shaping mandrel 102, so that the resulting intermediate tubular structure includes larger-diameter segments and smaller-diameter segments in an alternating sequence. Then, however, the intermediate tubular structure is drawn or radially contracted onto a constant diameter heat-set mandrel. Larger-diameter segments of the structure, as compared to smaller-diameter segments, are drawn longitudinally a greater distance to reduce their diameters to the diameter of the heat-set mandrel. Clamps may be used, particularly around the larger-diameter segments, to ensure that the intermediate tubular structure more closely conforms to the mandrel. Accordingly, the resulting constant-diameter stent appears similar to stent 26 in FIG. 4, with alternating segments having relatively high and relatively low strand crossing angles. The lower crossing angle segments of the finished stent correspond to the larger-diameter segments of the intermediate tubular structure.

FIGS. 17 and 18 illustrate initial stages of a process for fabricating a stent similar to stent 46 (FIG. 7). In FIG. 17, a first filament 108 from a spool 110, and a second filament 112 from a spool 114, are simultaneously wound onto a bobbin 116 to provide a paired-filament strand 118. Bobbin 116 is one of several (e.g. 36) bobbins loaded in a braiding apparatus 120 for simultaneous winding of the paired-filament strands onto a constant-diameter shaping mandrel 122.

In one specific example, each of filaments 108 and 112 is an Elgiloy alloy wire having a diameter of 0.14 mm. A stent formed of paired strands 118 has similar radial force and axial stiffness levels as a stent formed of the same number of single-wire strands at a 0.17 mm diameter, assuming the same strand crossing angle, which in this example is 130 degrees.

In a variant of this process, paired-filament strands, slightly twisted to provide a cable or rope, may be purchased in lieu of performing the bobbin loading step illustrated in FIG. 17.

In either event, after braiding the intermediate tubular structure is removed from the shaping mandrel and disposed on a constant-diameter heat-set mandrel preferably having the same diameter as the shaping mandrel, e.g. 22 mm. As before, the intermediate tubular structure is heated to a temperature sufficient to thermally impart the desired shape to the stent.

When removed from the heat-set mandrel, the stent has substantially the same strand crossing angle throughout its length, as well as the same levels of radially outward force and axial flexibility. At this stage, filaments 112 are cut at selected points along the length of the stent, corresponding to junctions between separate stent segments. Following cutting, each filament 112 is severed into alternating first and second filament segments. The first segments are removed from the stent to form segments along which strands 118 include only filaments 108, such as segments 52 of stent 46. The second filament segments remain in place, providing stent segments along which the strand incorporates both filaments 108 and 112.

Table 3 shows levels of radially outward force and axial stiffness in stents employing 36 strands of the Elgiloy alloy. Levels are indicated for the different segments, along which each strand includes two filaments and only one filament, respectively.

TABLE 3

| Filament | Radial | Longitudinal | Radial | Longitudinal |
|---|---|---|---|---|
| Diameters, | Pressure, | Stiffness, N/m | Pressure, | Stiffness, N/m |
| mm | Pa (2 Wires) | (2 Wires) | Pa (1 Wire) | (1 Wire) |
| 0.34 | 40952 | 456 | 20476 | 228 |
| 0.17 | 2560 | 28.6 | 1280 | 14.3 |
| 0.15 | 1552 | 17.4 | 776 | 8.7 |
| 0.12 | 636 | 7.0 | 318 | 3.5 |

Thus, the stent segments along which the strand incorporates two wires, as compared to the stent segments along which the strand has a single wire, exert twice the radially outward force and have twice the longitudinal stiffness, when a stent formed on a 22 diameter mandrel is radially compressed to a 20 mm diameter. As noted above, the two filaments of the strand can have different diameters if desired, to alter the relationship of the alternating stent segments as to both radial force and axial stiffness.

Table 4 shows levels of radially outward force and axial stiffness in stents constructed of a biostable polymer such as PET. Levels are given for stent segments along which the strands consist of single filaments, and for alternate segments in which the strands include two filaments of the given diameter. While not shown in the table, filaments of different diameters would provide cumulative radial pressure and longitudinal stiffness levels when present in the strand. For example, a stent segment along which the strands each incorporate one 0.35 mm diameter filament and one 0.4 mm diameter filament would exert a radial pressure of 1,520 Pa, and have a longitudinal stiffness of 16.6 N/m. In this example, the 36 polymeric strands are wound and interbraided on a 22 mm diameter shaping mandrel at a strand crossing angle of 130 degrees, and heat treated on a heat-set mandrel having the same diameter. As a result, the finished stent has a braid angle of 130 degrees.

TABLE 4

| | | | | Longitudinal |
|---|---|---|---|---|
| Filament | Radial | Longitudinal | Radial | Stiffness, N/m |
| Diameters, | Pressure, Pa | Stiffness, N/m | Pressure, Pa | (Single |
| mm | (2 Filaments) | (2 Filaments) | (1 Filament) | Filament) |
| 0.24 | 234 | 2.6 | 117 | 1.3 |
| 0.3 | 586 | 6.4 | 293 | 3.2 |
| 0.35 | 1108 | 12.2 | 554 | 6.1 |
| 0.4 | 1932 | 21 | 966 | 10.5 |
| 0.5 | 4928 | 52.4 | 2464 | 26.2 |

All radial pressure and longitudinal stiffness levels are produced under radial compression of the stent to a diameter of 20 mm. Based on Table 4, a stent segment in which the strands each consist of a pair of polymeric filaments most closely approximates the radial pressure and longitudinal stiffness levels of a stent segment with Elgiloy alloy strands (0.17 mm diameter) when the diameter of each filament is in the range of 0.35-0.40 mm.

As previously noted, the paired-filament strands making up stents like stent 46 can consist of filaments formed of different materials: e.g., a filament 54 composed of the Elgiloy alloy, and a filament 56 composed of a biostable polymer such as PET. In such cases stent fabrication is subject to a restriction not present when both filaments are the same. Conversely, certain fabricating options are available that cannot be employed when the filaments are the same.

The restriction applies to the thermal setting stage. In particular, metals like the Elgiloy alloy typically are thermally set at temperatures that not only exceed thermal setting temperatures of the biostable polymers, but also the melting temperatures of the biostable polymers. Accordingly, a braided intermediate tubular structure incorporating both the metal and polymeric filaments cannot simply be heat set on a heat-set mandrel as when all filaments are either metal or polymeric.

To overcome this problem, one alternative process begins with selecting, as the metal for filaments 54, a cobalt-chromium-molybdenum (CoCrMo) alloy containing less than about 5 weight percent nickel as the metallic filament material. Several such alloys are described in U.S. Pat. No. 5,891,091 (Stinson), assigned to the assignee of this application. Filaments composed of these alloys can be shaped by cold working, without heat treatment. Accordingly, after an intermediate tubular structure is disposed onto a heat-set mandrel as previously described, the temperature of intermediate structure is heat set by raising it only to the lower heat-set temperature of the polymeric biostable filaments, not to the much higher heat-set temperature of the metal filaments.

According to another suitable alternative process, the metallic filaments are thermally set, and thus tend to assume the desired helical shape, before they are combined with the biostable polymeric filaments. The polymeric filaments may likewise be preshaped, or alternatively may be thermally set after they are combined with the metallic filaments at the much lower heat-set temperatures that apply to the polymer. The preshaping proceeds as described in U.S. Pat. No. 5,758,562 (Thompson), assigned to the assignee of this application.

On the other hand, when filaments 54 and 56 are formed of different materials, alternative methods of selectively removing portions of filaments 56 can be employed, which are not available when the filaments are the same. FIG. 19 illustrates an intermediate tubular structure 124, following a heat-set stage. Throughout the length of the structure, helically wound and interbraided strands 126 are each composed of a metallic filament and a biostable polymeric filament. The tubular structure is disposed proximate a laser 128 that generates a laser beam 130, either in a continuous wave (CW) or pulsed mode. In either event, beam 130 is caused to selectively impinge upon the polymeric filaments at selected points along the length of the tubular stent.

The selected points can correspond to junctions between high radial force segments and low radial force segments, in which case filaments 56 are severed by laser ablation for later removal from intended low radial force segments. Alternatively, stent 124 can be moved axially relative to the laser beam, such that length portions of filaments 56 along intended low axial stiffness sections are completely removed by laser ablation. In either case, the laser ablation has a negligible impact on adjacent metallic filaments 54. To further ensure that laser ablation removes filaments only along intended lower axial stiffness segments, filaments 56 along the intended higher axial stiffness segments can be masked or shielded during the laser treatment.

The laser ablation processees can be automated, substantially reducing the fabrication cost compared to situations that require cutting the filaments.

In an alternative process, length portions of the polymeric filaments can be dissolved along the intended low axial stiffness segments, using a solution in which the metallic filaments remain stable. Again, it may be desired to mask or shield the polymeric filaments along intended high axial stiffness segments.

In yet another alternative similar to laser ablation, stent 124 is selectively, i.e. specifically along intended lower axial stiffness segments, subjected to heat sufficient to melt the polymeric filaments. Heat-reflective or heat-insulative shielding can be interposed between adjacent segments, to ensure that the polymeric filaments are melted only along the intended segments.

Thus in accordance with the present invention, stents, stent-grafts and other devices implantable in body vessels can be selectively varied along their lengths both as to axial stiffness and radial force, in each case to achieve an optimum combination of fixation and conformity with body vessel curvature. Alternatively or in addition, such devices can provide high initial levels of radial force and axial stiffness that gradually degrade in vivo. Depending on the fabrication approach, devices can be configured with segments that exceed neighboring segments as to both radial force and axial stiffness, or alternatively exceed neighboring segments as to radial force and axial flexibility. In helically wound devices, pitch and strand crossing angles can be kept lower, to reduce the degree of axial contraction that accompanies radial expansion.

What is claimed is:

1. A body insertable prosthesis, including:
a body insertable tubular structure including at least one flexible strand selectively formed to provide a plurality of discrete tubular segments including a first segment, a second segment spaced apart axially from the first segment, and a third segment disposed between the first and second segments;
wherein each of the segments has a nominal diameter when the tubular structure is in a relaxed state and is radially compressible against an elastic restoring force to a predetermined diameter;
wherein the at least one flexible strand further is selectively configured to provide first, second and third axial stiffness levels and first, second and third radial force levels along the first, second and third segments, respectively, when said segments are radially compressed to the predetermined diameter; and
wherein the third axial stiffness level is outside of a range of axial stiffness levels bound by the first and second axial stiffness levels.

2. The prosthesis of claim 1 wherein:
the first and second axial stiffness levels are substantially the same.

3. The prosthesis of claim 1 wherein:
the first and second axial stiffness levels are less than the third axial stiffness level.

4. The prosthesis of claim 1 wherein:
the first and second axial stiffness levels are higher than the third axial stiffness level.

5. The prosthesis of claim 1 wherein:
the at least one flexible strand includes a plurality of flexible strands helically wound in opposite directions to form multiple strand crossings defining strand crossing angles, including respective first, second and third strand crossing angles along the first, second and third segments, respectively; and
the third strand crossing angle is outside of a range of strand crossing angles bound by the first and second strand crossing angles.

6. The prosthesis of claim 5 wherein:
the third strand crossing angle is larger than the first and second strand crossing angles, and the third axial stiffness level is less than the first and second axial stiffness levels.

7. The prosthesis of claim 5 wherein:
the third strand crossing angle is smaller than the first and second strand crossing angles, and the third axial stiffness level is higher than the first and second axial stiffness levels.

8. The prosthesis of claim 1 wherein:
the third radial force level is outside of a range of radial force levels bound by the first and second radial force levels.

9. The prosthesis of claim 8 wherein:
the third axial stiffness level is higher than the first and second axial stiffness levels, and the third radial force level is higher than the first and second radial force levels.

10. The prosthesis of claim 8 wherein:
the third axial stiffness level is higher than the first and second axial stiffness levels, and the third radial force level is lower than the first and second radial force levels.

11. The prosthesis of claim 1 wherein:
the at least one strand incorporates a first number of filaments along a first region of the tubular structure and incorporates a second number of filaments, less than the first number, along a second region of the tubular structure; and
one of the first and second regions includes the first and second segments, and the other of said regions includes the third segment.

12. The prosthesis of claim 11 wherein:
the first region includes the first and second segments, whereby the first and second axial stiffness levels are higher than the third axial stiffness level and the first and second radial force levels are higher than the third radial force level.

13. The prosthesis of claim 11 wherein:
the first region includes the third segment, whereby the third axial stiffness level is higher than the first and second axial stiffness levels, and the third radial force level is higher than the first and second radial force levels.

14. The prosthesis of claim 1 wherein:
the tubular structure consists essentially of an alternating series of segments having relatively high axial stiffness levels and segments having relatively low axial stiffness levels, said alternating series including the first, second and third segments.

15. The prosthesis of claim 14 wherein:
each of the segments of the alternating series has an axial length of at least about 1 cm.

16. The prosthesis of claim 1 wherein:
the at least one strand includes a first set of flexible strands spanning substantially the length of the tubular structure, and a second set of flexible strands extending along a first region of the tubular structure to provide a higher axial stiffness and a higher radial force along the first region, and a lower axial stiffness and lower radial force along a second region that does not include the second set of flexible strands; and
one of the first and second regions includes the first and second segments, and the other of said regions includes the third segment.

17. The prosthesis of claim 1 wherein:
the first, second and third segments have substantially the same nominal diameters.

18. The prosthesis of claim 1 wherein:
the first and second segments have respective nominal diameters that are substantially the same, and different than a nominal diameter of the third segment.

19. A body insertable device including:
a body insertable tubular structure including at least one flexible strand selectively formed to define a plurality of discrete tubular regions of the tubular structure including at least a first region and a second region;
wherein each of the regions has a nominal diameter when in a relaxed state and is compressible against an elastic restoring force to a predetermined diameter less than its nominal diameter; and
wherein the at least one strand incorporates a first number of filaments along the first region and incorporates a second number of filaments along the second region, the second number being less than the first number whereby the first region has a first axial stiffness level higher than a second axial stiffness level of the second region.

20. The device of claim 19 wherein:
the at least one strand along the first region incorporates first and second different types of filaments, and the strand along the second region incorporates only the first filament type.

21. The device of claim 20 wherein:
the first filament type is selected from the group of filament types consisting of: metallic filaments and biostable non-metallic filaments; and the second filament type is selected from the group of filament types consisting of: metallic filaments, biostable non-metallic filaments, and biodegradable filaments.

22. The device of claim 19 wherein:
the at least one strand comprises a cable incorporating at least two filaments along the first region.

23. The device of claim 19 wherein:
the tubular structure includes an alternating series of first tubular segments having relatively high axial stiffness levels and second tubular segments having relatively low axial stiffness levels, wherein the first tubular region includes the first tubular segments and the second tubular region includes the second tubular segments.

24. The device of claim 23 wherein:
the tubular structure includes, at first and second opposite ends thereof, end segments selected from the group of end segments consisting of: two first segments; two second segments; and a first segment and a second segment.

25. A prosthesis insertable into body lumens with natural curvature, including:
a body insertable tubular wall incorporating an alternating sequence of first and second tubular wall segments including at least three of the wall segments, each of the wall segments having a nominal diameter when in a relaxed state and being radially compressible against an elastic restoring force to a predetermined diameter;
wherein the wall segments when radially compressed to the predetermined diameter have respective axial stiffness levels, with each of the first tubular wall segments having a relatively high axial stiffness level, and with each of the second tubular wall segments having an axial stiffness level lower than that of the first tubular wall segments whereby the second tubular wall segments, as compared to the first tubular wall segments, more readily conform to a curvature of a body lumen in which the tubular wall is deployed.

26. The prosthesis of claim 25 wherein:
all of the first tubular wall segments have substantially the same axial stiffness, and all of the second tubular wall segments have substantially the same axial stiffness.

27. The prosthesis of claim 26 wherein:
the body insertable tubular wall is composed of at least one flexible strand selectively formed to provide the alternating first and second tubular wall segments.

28. The prosthesis of claim 27 wherein:
the at least one flexible strand includes a plurality of flexible strands helically wound in opposite directions to form multiple strand crossings defining strand crossing angles, and wherein the strand crossing angles along the second tubular wall segments are larger than the strand crossing angles along the first tubular wall segments.

29. The prosthesis of claim 25 wherein:
the tubular wall segments have respective radial force levels when radially compressed to the predetermined diameter, and the radial force levels of the first tubular wall segments are higher than the radial force levels of the second tubular wall segments.

30. The prosthesis of claim 25 wherein:
the tubular wall segments have respective radial force levels when radially compressed to the predetermined diameter, and the radial force levels of the first tubular wall segments are lower than the radial force levels of the second tubular wall segments.

31. The prosthesis of claim 27 wherein:
the at least one flexible strand incorporates a first number of filaments along each of the first tubular wall segments and a second number of filaments along each second tubular wall segment, wherein the second number is less than the first number.

32. A body insertable stent, including:
a stent structure formed of at least one flexible composite strand and adapted for deployment at a site within a body lumen to apply a radially outward force against surrounding tissue at the site;
wherein the composite strand includes at least one biostable filament and at least one bioabsorbable filament and is adapted to apply the radially outward force initially upon deployment in situ at a first level due to a combination of the at least one biostable filament and the at least one bioabsorbable filament; and
wherein the at least one bioabsorbable filament is absorbable in situ, thereby to reduce the radially outward force toward a second level due to the at least one biostable filament alone.

33. The stent of claim 32 wherein:
the stent structure along its length includes a plurality of discrete regions including a first region along which the at least one strand incorporates the at least one biostable filament and the at least one bioabsorbable filament, and a second region along which the at least one strand incorporates only the at least one biostable filament.

34. The stent of claim 32 wherein:
the at least one composite strand includes a plurality of the biostable filaments, and a plurality of the bioabsorbable filaments.

35. The stent of claim 32 wherein:
the at least one composite strand includes a plurality of the strands helically wound in opposite directions to form multiple strand crossings defining strand crossing angles.

36. The stent of claim 32 wherein:
the at least one biostable filament comprises a non-metallic filament.

37. A process for fabricating a body insertable prosthesis adapted to apply outward radial forces that vary along the prosthesis length, including:
providing at least one flexible, biocompatible and thermally formable strand;
winding the at least one flexible strand onto a substantially constant diameter cylindrical shaping mandrel, and altering a pitch at least once during winding to form a tubular prosthesis structure having a selected shape in which the at least one strand is wound at a first pitch along a first region of the structure, and wound at a second pitch different from the first pitch along a second region of the structure; and
while maintaining the at least one strand in the selected shape, heating the tubular structure to a temperature sufficient to thermally impart the selected shape to the tubular structure.

38. The process of claim 37 wherein:
said winding comprises winding at least first and second flexible strands helically in opposite directions to form multiple intersections of the strands, wherein the intersecting strands a define different first and second strand crossing angles along the first and second regions, respectively.

39. The process of claim 37 further including:
after said winding, removing the tubular structure from the shaping mandrel, and placing the tubular structure onto a substantially constant diameter heat-set mandrel; and
wherein said heating the tubular structure comprises heating the heat-set mandrel.

40. A process for fabricating a body insertable prosthesis, including:
winding at least one flexible, biocompatible and thermally formable strand onto a substantially constant diameter shaping mandrel at a substantially uniform pitch, to form a tubular structure;
removing the tubular structure from the shaping mandrel, placing the tubular structure onto a heat-set mandrel having a plurality of mandrel segments with different diameters;
causing the tubular structure to conform to the heat-set mandrel and thus assume a selected shape in which the tubular structure has a first region with a first diameter, a second region with a second diameter, and an intermediate region between the first and second regions and having a third diameter outside of a range of diameters bound by the first and second region diameters.; and
with the tubular structure conforming to the heat-set mandrel, heating the heat-set mandrel sufficiently to thermally impart the selected shape to the tubular structure.

41. The process of claim 40 wherein:
the winding of the at least one strand comprises helically winding at least first and second strands in opposite directions to form multiple intersections of the strands.

42. A process for fabricating a body insertable prosthesis, including:
winding at least one flexible, biocompatible and thermally formable strand onto a shaping mandrel having at least a first region with a first diameter and a second region disposed axially of the first region and having a second diameter different than the first diameter, to form a tubular structure having an initial shape;
removing the tubular structure from the shaping mandrel, and disposing the tubular structure along and in surrounding relation to a heat-set mandrel;
causing the tubular structure to substantially conform to the heat-set mandrel, thereby to assume a selected shape; and
with the tubular structure in the selected shape, heating the tubular structure to a temperature sufficient to thermally impart the selected shape to the tubular structure and thereby form in the tubular structure first and second segments corresponding respectively to portions of the tubular structure previously disposed along the first and second regions of the shaping mandrel, said first and second segments being adapted to exert respective first and second different levels of radially outward force when the tubular structure is radially compressed to a predetermined diameter.

43. The process of claim 42 wherein:
the heat-set mandrel has a substantially constant diameter, whereby the tubular structure in the selected shape has a substantially constant diameter.

44. The process of claim 42 wherein:
the heat-set mandrel has first and second heat-set mandrel regions with different diameters corresponding to the first and second regions of the shaping mandrel, whereby the first and second segments of the tubular structure in the selected shape have respective first and second different diameters.

45. A process for fabricating a body insertable prosthesis with segments that differ in axial stiffness and radial force, including:
providing a flexible strand that is a composite of at least two body compatible filaments;
selectively winding the strand to form a tubular structure with a selected shape; and
selectively removing at least one of the filaments from the at least one strand along a predetermined axially extended region of the tubular structure, whereby the tubular structure along the predetermined region has a reduced axial stiffness level and a reduced radial force level as compared to a remaining region of the tubular structure.

46. The process of claim 45 wherein:
selectively winding the strand comprises winding the strand onto a substantially constant diameter shaping mandrel.

47. The process of claim 45 further including:
after selectively winding the strand and while maintaining the tubular structure in the selected shape, heating the tubular structure to a temperature sufficient to thermally impart the selected shape to the tubular structure.

48. The process of claim 45 wherein:
selectively removing at least one of the filaments comprises cutting the at least one filament at a plurality of selected points along the strand to separate a length of the at least one filament extending along the predetermined region, then removing the separated length of the filament from the tubular structure.

49. The process of claim 45 wherein:
said filaments include at least one first filament having a first melting temperature and at least one second filament having a second melting temperature lower than the first melting temperature; and
wherein removing at least one of the filaments comprises heating the tubular structure at selected points to a temperature lower than the first temperature and higher than the second temperature.

50. The process of claim 49 wherein:
the heating comprises laser ablation of the at least one second filament.

51. The process of claim 50 wherein:
the at least one strand includes at least one substantially insoluble first filament and at least one soluble second filament; and
wherein the removing of at least one of the filaments comprises dissolving the at least one second filament along the selected region.
